(19) United States
(12) Patent Application Publication
(10) Pub. No.: US 2010/0297604 A1
(43) Pub. Date: Nov. 25, 2010

(54) METHODS AND REAGENTS FOR VIRUS ISOLATION AND DETECTION

(76) Inventors: Xing-Xiang Li, Vienna, VA (US); Tianxin Wang, Columbia, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/304,092

(22) PCT Filed: Jun. 11, 2007
(86) PCT No.: PCT/US07/13636

§ 371 Date: Aug. 3, 2010

Related U.S. Application Data
(60) Provisional application No. 60/812,093, filed on Jun. 9, 2006.

(51) Int. Cl. C12Q 1/70; C12N 7/02; C12Q 1/68; C08B 37/00; C08B 37/10; C08F 28/02; B01D 15/08

(52) U.S. Cl. 435/5; 435/239; 435/6; 536/53; 536/21; 525/328.5; 210/198.2

(57) ABSTRACT

The present invention relates to reagents and methods used in virus isolation and analysis.

This application claims the benefit of U.S. Provisional Application Ser. No. 60/812,093, filed Jun. 9, 2006, the entire disclosure of which is hereby incorporated by reference.

This invention was supported by a grant from the US government.

### FIELD OF THE INVENTION

The present invention relates to methods and reagents for virus isolation and detection.

### BACKGROUND

Human infection by viruses is one of the major health problems facing the mankind. Human immunodeficiency virus (HIV) has resulted in some 40 million deaths so far. Although an array of pharmaceutical drugs are available now, the use of these drugs requires the monitoring of the efficacy of the drugs by measuring the virus concentration in blood samples, commonly known as HIV viral load. HIV viral load is normally determined by measuring the viral RNA concentration in the blood samples (serum or plasma). A number of methods have been developed for measuring viral RNA concentration with the most commonly used method being reverse transcription polymerase chain reaction (RT-PCR).

The most cumbersome step to carry out RT-PCR for HIV detection is the extraction of RNA from the sample, particularly for those samples with very low viral concentration, e.g., less than 100 RNA copies/mL. For the detection of low HIV viral load, the samples are often centrifuged to enrich the viruses using an ultracentrifugation process, which requires an expensive ultracentrifuge and a long centrifugation time (approximately 2 hours). It would greatly simplify HIV RNA detection if an efficient and inexpensive approach can be developed to replace the ultracentrifugation process. Indeed, HIV specific antibody coupled with biotin had been used for this purpose. The biotinylated HIV antibody is first incubated with the HIV-containing samples to label the virus with biotin. The HIV viruses are then captured with avidin-coated magnetic particles. The drawbacks with this approach are that the biotinylated HIV antibody has to compete with the human antibodies for the same antigen and that both reagents are expensive. Therefore, it would be quite useful to have a simpler and less expensive approach.

Influenza viruses are another example where a virus isolation reagent would be useful for a number of applications. Influenza is a constant and serious threat to public health. Each year, Influenza epidemic leads to 200,000 hospitalizations and 36,000 deaths in the United States. An influenza pandemic could lead to far greater number of deaths and economic impact. The 1918 influenza pandemic, for example, killed 20-40 million people in the world and more than 500,000 people in the United States. As medical science advances, there are several drugs that are available for treatment or prophylaxis of influenza A and B. However, the prerequisite for effective treatment or prevention control is rapid, sensitive and specific detection of the viruses at an early stage of individual infection or of an outbreak.

Conventional method for influenza virus detection involves first viral culture of a nasal wash, throat swab specimen, tracheal aspirate secretions, bronchial lavages, or lung tissue. The virus in cultures usually is detected between day 2 and day 5 by performing hemabsorption test, which is normally followed by detection with an immunofluorescence assay (IFA) using type specific antibodies. This method takes a long time, is cumbersome and, consequently, is not appropriate for point-of-care use, e.g., for aiding the physicians in making therapeutic decisions.

Speedier diagnostic immunoassays have been developed for point-of-care use. However, these tests generally lack sufficient sensitivity, which greatly hinder their usefulness as a rapid diagnostic tool. For example, a recent study, which compared three commercial tests, showed that the sensitivity ranges from 39% to 76%.

One of the major problems with influenza virus detection is the lack of a rapid and efficient virus isolation and concentration reagent or method, which is compatible for downstream detection. This is because the clinical specimens (nasal wash and throat swab wash solution) are often in large volume and in complex matrix. Current methods of sample processing for point-of-care use diagnostic tests often involves mixing the sample with a diluent solution followed by filtration to remove large debris. This method does not concentrate the viral particles, which may significantly reduce the sensitivity of virus detection.

Recent outbreaks of the highly pathogenic avian flu virus H5N1 subtype and occasional human infection by this virus has heightened the concern that this virus, or other avian flu viruses, may turn into a pandemic human influenza virus. Surveillance on these viruses in animal population and in their living environment, e.g., water system in which an infected duck population live is an important aspect for preventing and monitoring the spread of these viruses. Current animal surveillance method involves eluting viruses from tracheal or cloacal samples into a storage buffer, which is then used for inoculating eggs or culture cells. The viruses eluted into the storage buffer is highly diluted and contaminated with substances that may inhibit virus growth. These deficiencies in current animal surveillance approaches may contribute in part to the low virus isolation rate in an infected bird population.

Therefore, there is an urgent need for a reagent or method that can be used for rapid and efficient isolation and concentration of influenza virus from clinical samples, animal samples and environmental samples. Preferably, this reagent or method is compatible with virus detection.

Another important application of purified viruses is for producing vaccines, therapeutics as in gene-therapy, and antigens for use in diagnostic tests. For example, current influenza vaccine production is accomplished using embryonated eggs. Influenza viruses grown in eggs are normally purified with the use of zonal ultracentrifugation, a lengthy process that requires expensive equipment. In addition, influenza vaccine production using embryonated eggs takes a long period of time and, consequently, not appropriate for the production of pandemic influenza vaccine. Cultured cells can be used to grow influenza viruses, including those strains with pandemic potential. A technology that enables efficient and economical influenza virus purification from cultured cells is necessary for producing influenza vaccine using cultured cells.

Virus purification is also necessary during research, development or production of therapeutics that use virus as a vector such as those for gene therapy. Moreover, antigens derived from purified viruses, such as those from cultured HIV viruses, can also be used to make diagnostic tests. Therefore, a virus purification technology is highly desirable for these applications as well.

Yet another application for the virus capture reagent or reagents is for removing viruses during the manufacturing of biopharmaceuticals, including human plasma derivatives, e.g., immunoglobulin. Different virus capture reagents can be used in combination in order to remove a broad spectrum of viruses.

### PRESENT INVENTION

The present invention provides a method for isolating and concentrating viruses in clinical, animal or environmental samples as well a method for the detection of them. The isolated viruses may be used for extracting nucleic acids, antigens or other viral components, which can be used for the detection of the viruses using a number of means, or for culturing in an appropriate culture system. The present invention also teaches a method for purifying virus for the purpose of producing vaccine, therapeutics, and antigens for diagnostics. Moreover, the present invention further provides a method for removing potentially contaminated virus from a biopharmaceutical during manufacturing.

These methods use a reagent that is composed of a solid support coupled with one or more chemicals, which have a binding affinity for the viruses to be isolated. The solid support could be a column, a membrane, a glass fiber, a particle, or any other appropriate surface, which contains appropriate surface properties either for direct coupling of the binding chemical or for coupling after modification.

In certain embodiments, the preferred solid support is magnetic particles for several reasons. First, commercial sources of magnetic particles with functionalized surface chemistry are readily available. Second, the magnetic particles can be efficiently and rapidly concentrated using a simple magnet. Finally, magnetic particles bound with influenza virus can be directly used for magnetism-based detection such as the BARC system, as described in examples for influenza detection. Preferred size of magnetic particle is between 50 nanometers (nm) to 100 micrometers (μm) in diameter. Many vendors (e.g. Bangslabs Inc, Spherotech, Inc. Seradyn, Inc.) provide magnetic particles suitable for current invention.

A virus binding chemical (or functional groups) should have two properties: 1) there is an affinity for the virus to be isolated and 2) the affinity for the virus remains after the chemical (or functional groups) is modified or coupled to a solid support. The affinity for the virus of the binding chemical is preferred to be selective. None selective binding chemical can also be used since specific methods, e.g., polymerase chain reaction (PCR) and antibody-based assays can be used for subsequent detection. The binding chemicals can be a pharmaceutical drug that specifically binds to virus surface or membrane protein, e.g., oseltamivir that binds to neuraminidase of influenza viruses. They can also be those that rely on relatively non specific charge-based binding between the chemical and viruses (e.g. ion exchange resin, beads having positively charged or negatively charged groups on their surface, an example of positively charged bead is bead having quaternary amine groups on its surface and an example of negatively charged bead is bead having sulfonic groups or sulfate group or carboxyl groups on its surface).

There are numerous methods for coupling a chemical to solid support. These methods are readily available from scientific journals, vendors that provide coupling reagents, or relevant websites. For example, chemicals containing a primary amine can be coupled to a solid support that is functionalized with a carboxyl group through the formation of amide bond; the formation of amide bond between the amine and carboxyl group is normally catalyzed with EDC [1-(3-dimethylaminopropyl)-3-ethylcarbodiimide hydrochloride] or other carbodiimide. Virus binding chemicals may need to be appropriately modified or derivatized to introduce a functional group that can be used for coupling while at the same time the modification or derivatization does not inactivate the virus binding activity.

It is understood that the binding chemicals can be chemically or naturally coupled to another moiety that can be subsequently coupled to a solid support. One example is the proteins extracted from erythrocyte membrane, which contains glycoproteins with sialic acid; these protein extracts can be coupled to a solid support that can be subsequently used for influenza virus isolation.

The method for virus isolation using the virus isolation reagent normally, but not always, consists of the following steps: The samples for virus isolation are first mixed with an optimal amount of virus isolation reagent under appropriate conditions (e.g., temperature, incubation duration etc.). The virus isolation reagent is then removed from the sample solution using an appropriate means. For example, if magnetic particles are used as the solid support, one can use a magnet to attract the reagent towards the test tube wall and remove the supernatant. The reagents can then be washed several times with an appropriate solution such as phosphate-buffered saline (PBS). Now the viruses are enriched on the surface of the reagent.

The isolated viruses can be detected by a number of methods, e.g., nucleic acid detection, antigen detection and viral enzyme detection. Depending on the method of detection, the captured viruses may be lysed with an appropriate solution as taught in Example 1. It is understood that the solutions for dissolving the viruses may need to be optimized or that existing solutions or methods can be used. In certain embodiments, the bound viruses may be eluted with the free binding chemicals. The viruses eluted in this manner may still be intact and be useful for culture isolation as described in Example 1.

The current invention also provides a convenient method for virus detection in a sample. The method consists of the following steps: The samples are first mixed with an optimal amount of virus isolation reagent described above under appropriate conditions (e.g., temperature, pH, incubation duration etc.). The virus isolation reagent is then removed from the sample solution using an appropriate means. For example, one can use a magnet to attract the reagent towards the test tube wall and remove the supernatant if the reagent is magnetic particle. Optionally, the reagents can then be washed several times with an appropriate solution such as phosphate-buffered saline (PBS). Now the viruses are enriched on the surface of the reagent. The isolated virus can be detected directly by PCR method without nucleic acid extraction step. In certain embodiments, the captured virus may be lysed with an appropriate solution such as taught in Example 1 and PCR is performed directly after the lysis step is carried out. This method greatly simplified the overall operation compared with the current methods, which involve tedious nucleic acid isolation steps.

It is understood that the scope of the present invention includes, but is not limited to, the use of virus isolation methods disclosed in the present invention, or the variations of such methods, for virus purification in research, development or production of vaccines, therapeutic agents, and diagnostic agents such as viral antigens.

For virus purification for the purpose of producing vaccines, therapeutic agents or diagnostic agents, the virus affinity chemical is first coupled to a solid support. In preferred embodiments, the resulting solid support is packed into a column. The column contains at least 0.05 mL, at least 0.1 mL, at least 0.5 mL, at least 5 mL, at least 50 mL, at least 500 mL or at least 1000 mL of the solid support coated with a virus binding chemical. It is understood that appropriate solid support, e.g., the size and surface property of the solid support, is preferred for use in a column so that appropriate flow rate is permitted. It is preferred that the solid support, e.g., microparticles, is large enough to allow the virus freely pass through unless it is coupled with the affinity chemical.

For purification of a virus, the virus containing solution, e.g., a culture medium containing influenza virus, is passed through the column. After washing with appropriate solution, e.g., PBS buffer, the bound virus is eluted. A number of methods can be used to elute the bound virus. For example, low pH (e.g., 2.4) buffer can be used to disrupt the binding between the virus and the affinity ligand on the solid support, thereby releasing the bound virus. Alternatively, a solution containing excessive amounts of the binding ligand is passed through the column to release the bound virus via competition binding. Other appropriate elution methods include, but are not limited to, physical methods such as heat, and elevated salt concentration.

It is further understood that the scope of the present invention includes, but is not limited to, the use of virus isolation methods disclosed in the present invention, or the variations of these methods, for virus removal or clearance during the manufacturing of biopharmaceuticals, including human plasma derivatives.

Aspects of the instant invention include:

1. A viral-particle isolation reagent, comprising:

(a) a virus-particle binding agent and (b) a solid support, wherein said binding agent is coupled to said solid support and is capable of specifically binding to a binding partner present in a virus particle.

2. A method for isolating a virus, comprising:

contacting virus particles with a viral-particle-isolation reagent under conditions effective for said reagent to specifically bind to said virus particles, wherein said reagent comprises a virus-particle-binding agent which is coupled to a solid support and which is capable of specifically binding to a binding partner present in said virus particles.

3. A method of aspect 2, wherein said virus-particle-binding-agent comprises polysialic acid or a derivative thereof.
4. A method of aspect 2, wherein said virus-particle-binding agent is capable of specifically binding to a virus particle neuraminidase protein.
5. A method of aspect 2, wherein said virus particle is present in a body fluid sample.
6. A method of aspect 2, wherein said virus particle is present in an environmental sample.
7. A method of aspect 2, further comprising eluting said virus particles from said reagent, and detecting neuraminidase activity present in said virus particles.
8. A method of aspect 2, further comprising contacting said reagent with a viral binding partner capable of specifically binding to said viral particle, wherein said binding partner is coupled to a solid substrate which is utilized to capture said reagent bound to said virus.
9. A method of aspect 8, wherein the solid support is a magnetic particle, and the presence of viral particles is determined by detecting the presence of magnetic material specifically bound to said binding partner.
10. An HIV capture reagent, comprising

(a) a polyanion and (b) a solid support, wherein said polyanion is coupled to said solid support and is capable of specifically binding to a surface binding partner on an HIV viral particle.

11. An HIV capture reagent of aspect 10, wherein said polyanion is a copolymer of maleic acid and styrenesulfonic acid, a polymer of polyvinyl phthalate sulfate, a sulfated polysaccharide, polyvinylsulfate, and polyanethole sulfonate, their copolymers with acrylic acids and salts thereof.
12. An HIV capture reagent of aspect 11, wherein said sulfated polysaccharide is curdlan sulfate, dextrin sulfate, fucoidan, and pentosan polysulfate, dextran sulfate, heparin, heparin sulfate, or carrageenan.
13. A Hepatitis virus capture reagent, comprising

(a) a polyanion and (b) a solid support, wherein said polyanion is coupled to said solid support and is capable of specifically binding to a surface binding partner on a Hepatitis viral particle.

14. A Hepatitis virus capture reagent of aspect 13, wherein said polyanion is heparin sulfate.
15. A Hepatitis virus capture reagent of aspect 14, wherein said Hepatitis viral particle is Hepatitis C virus.
16. A Hepatitis virus capture reagent of aspect 14, wherein said Hepatitis viral particle is Hepatitis B virus or Hepatitis A virus.
17. A Hepatitis virus capture reagent of aspect 13, wherein said polyanion is curdlan sulfate, dextrin sulfate, fucoidan, and pentosan polysulfate, dextran sulfate, heparin, heparin sulfate, carrageenan, copolymer of maleic acid and styrenesulfonic acid, polymer of polyvinyl phthalate sulfate, sulfated polysaccharide, polyvinylsulfate, or polyanethole sulfonate, or a copolymer thereof with acrylic acids or a salt thereof.
18. A method of purifying a virus from a biological or an environmental solution comprising treating said solution with a reagent of aspect 1.
19. A method of removing a virus from a biological or an environmental solution comprising treating said solution with a reagent of aspect 1.
20. A column packed with a reagent of aspect 1.
21. A method of purifying a virus from a biological or an environmental solution comprising (a) passing the virus-containing solution through a column of aspect 20, (b) washing the column to remove unwanted materials, and (c) eluting the bound virus.
22. A method of detecting a virus from a biological or an environmental specimen comprising (a) treating said specimen with a reagent of aspect 1 and (b) releasing the nucleic acid from the virus bound to the said reagent and (c) directly detecting the released nucleic acid using polymerase chain reaction technique without nucleic acid extraction step.
23. A method of detecting and recovering a virus from a biological or an environmental specimen comprising (a) treating said specimen with a reagent of aspect 1, (b) directly contacting the reagent resulted from step (a) without releasing the virus with cultured cells or tissue suitable for propagation of the said virus, and (c) detecting and recovering the said virus from the culture.

The entire disclosures of all applications, patents and publications, cited above and below, are hereby incorporated by reference.

### BRIEF DESCRIPTION OF THE DRAWINGS

Various features and attendant advantages of the present invention will be more fully appreciated as the same becomes better understood when considered in conjunction with the accompanying drawings, in which like reference characters designate the same or similar parts throughout the several views, and wherein:

FIG. 1 shows schematic illustration of an influenza virus binding reagent.

(A) A schematic illustration of an influenza virus binding reagent, which is composed of a magnetic particle coupled with polysialic acid.
(B) A schematic illustration of a polysialic acid with 2, 8 linkage. This chemical can be coupled to a solid phase support to make a reagent for influenza virus isolation and concentration. It can also be used for SV40 virus isolation.

FIG. 2 shows an example of C-glycoside of N-acetylneuraminic acid coated magnetic particle. The FIGURE illustrates possible variation and derivatization of sialic acid as the influenza virus binding chemical.

FIG. 3 shows a photograph of a gel electrophoresis of PCR products resulted from an experiment, in which the influenza virus isolation reagent was used to isolate and concentrate influenza viruses in 1 mL PBS buffer. The isolated viruses were extracted for genomic RNA, which were used for RT-PCR followed with another round of nested PCR. The influenza virus binding reagent used in this experiment was magnetic particles coated with 2,8-polysialic acid.

FIG. 4 shows photograph of a gel electrophoresis of PCR products resulted from an experiment, in which the viruses were first isolated with the influenza isolation reagent and then eluted with free polysialic acid at 1000 ng/mL. This experiment used more influenza viruses so that the PCR product could be visualized after a single round of RT-PCR. The influenza virus binding reagent used in this experiment was magnetic particles coated with 2,8-polysialic acid.

FIG. 5 shows a schematic illustration showing one example of chemical, copolymers of maleic acid and styrenesulfonic acid (PSMA). This chemical can be coupled to a solid phase support for isolation and concentration of HIV, HBV and other viruses.

FIG. 6 shows results of gel electrophoresis

Lanes 1-4 are for samples with input HIV-1 viruses at 0, 50, 100 or 500 RNA copies/mL, respectively. Lane 5: a positive control with HIV-1 RNA isolated using a commercial nucleic acid extraction kit. Lane 6: DNA Marker/Ladder

FIG. 7 shows results of gel electrophoresis

Lane 1: DNA marker (kb Ladder); lane 2 negative control; lane 3: 100 IU HBV positive control; lane 4: 25 IU HBV/ml human serum; lane 5: 50 IU HBV/ml human serum; lane 6: 100 IU HBV/ml human serum; lane 7: 500 IU HBV/ml human serum; lane 8: 1000 IU HBV/ml human serum.

### DEFINITIONS

To facilitate understanding of the invention, a number of terms are defined below.

The terms “virus,” “virion” and “viral particle” are and can be used interchangeably, and include all viruses (e.g., enveloped and non-enveloped) which express proteins on their surface, including envelope proteins, coat proteins and cellular membrane proteins, as well as “naked viruses which lack such surface proteins but which can be modified to include them (e.g., by insertion of the proteins into the outer lipid bilayer of the virus). Such viruses include for example, but are not limited to, retroviruses and DNA viruses.

The term “viral particle” as used herein refers to the fully or partially assembled capsid of a virus. A viral particle may or may not contain nucleic acid.

The term “capsid” or “viral capsid” as used herein refers to the protein coat that surrounds the viral nucleic acid in a wild-type virus. Viral capsids have interior surfaces and exterior surfaces. The interior surface of a viral capsid is the surface that is normally exposed to the viral nucleic acid. The exterior surface of a viral capsid is the surface that is generally exposed to the environment.

Viral particles which can be isolated by the methods of the present invention include a broad variety of viruses. For example, the virus can be an “enveloped virus” which are a class of viruses whose core is surrounded by the viral envelope. The viral envelope is usually a lipid bilayer produced upon budding from the packaging cell's plasma membrane and also comprises one or more proteins encoded by viral genes referred to herein as “viral envelope proteins.” The term “viral envelope protein” refers to a protein in the viral envelope which interacts with a specific cellular protein to determine the target cell range of the virus. “Viral envelope proteins” include both naturally occurring (i.e., native) envelope proteins and functional derivatives thereof, as well as synthetic forms thereof (e.g., recombinantly produced viral envelope proteins).

As is well known in the art, altering the viral envelope (env) gene or its gene product can be used to manipulate the target cell range of the virus. For example, replacing the env gene of one virus with the env gene of another virus (referred to as “pseudotyping”) can extend the host range of a virus. Thus, a “pseudotyped virus” refers to a virus having an envelope protein that is from a virus other than the virus from which the viral genome is derived. For example, the envelope protein can be from a retrovirus of a species different from the retrovirus from which the RNA viral genome is derived or from a non-retroviral virus.

The present invention also can be used to isolate “non-enveloped” viral particles. Non-enveloped viruses have an external structure primarily composed of a “viral coat protein” encoded by viral genes. Accordingly, as used herein, the term “viral coat protein” refers to proteins which create the tightly assembled structure of the protective shell for non-enveloped viruses and prevent degradation of the genome by environmental factors.

In addition, the present invention can be used to isolate “naked virions.” As used herein, the term “naked virion” refers to virions produced by membrane budding, e.g., from packaging cells, in the absence of expressed envelope protein. However, naked virions contain cell-specific proteins in the lipid membrane referred to herein as “cellular membrane proteins.” As used herein, the term “semi-synthetic viral vectors” refers to a viral particle produced by adding a separately produced recombinant envelope protein, with or without pseudotyping, to a naked virion.

The term “polysialic acid” refers to a polymer of sialic acid. As is known in the art, sialic acid is a generic term for the N- or O-substituted derivatives of neuraminic acid, a nine-carbon monosaccharide. It is also the name for the most common member of this group, N-acetylneuraminic acid (Neu5Ac or NANA). The amino group bears either an acetyl or a glycolyl group. The hydroxyl substituents may be varied, for example, to include one or more acetyl, lactyl, methyl, sulfate and/or phosphate groups.

As described hereinbefore, the virus capture reagent may comprise a polyanion which comprises maleic acid, styrenesulfonic acid or a copolymer thereof, a polymer of polyvinyl phthalate sulfate, a sulfated polysaccharide (for e.g., curdlan sulfate, dextrin sulfate, fucoidan, and pentosan polysulfate, dextran sulfate, heparin, heparin sulfate, or carrageenan), polyvinylsulfate, and polyanethole sulfonate, their copolymers with acrylic acids and salts thereof.

The term “derivative” as used herein refers to a modified, usually chemically modified mono or polysaccharide compounds. Derivatives of polysialic acid (PSA) are exemplified in US Patent Application Nos. 2006/0270830 and 2007/0010482, which are incorporated by reference in their entirety.

The term “biological sample” refers to any sample isolated from an organism and includes, but is not limited to, any fluid, cellular, tissue, and/or organ obtained from such organisms. In one aspect, the biological sample includes a nasal wash specimen, a throat swab, a blood sample, a stool sample, a urine sample, a mucus specimen, or a sputum sample. In other embodiments, the biological sample includes sections of tissues such as frozen sections taken for histological purposes, including animal and plant tissue sections. Most preferably, the biological sample is isolated from a human source. In still other embodiments, the biological sample refers to materials derived from cell or tissue culture.

The term “environmental sample” refers to any sample isolated from environmental sources, and includes, but is not limited to a soil sample, an air sample, or a water sample.

Without further elaboration, it is believed that one skilled in the art can, using the preceding description, utilize the following invention to its fullest extent. The following specific preferred embodiments are, therefore, to be construed as merely illustrative, and not limitative of the remainder of the disclosure in any way whatsoever.

In the forgoing and in the following examples, all temperatures are set forth uncorrected in degrees Celsius and, all parts and percentages are by volume, unless otherwise indicated.

### EXAMPLES

The invention will be explained below with reference to the following non-limiting examples.

### Example 1

### Influenza Virus Isolation and Detection

Influenza viruses contain several surface membrane proteins, including hemagglutinin (HA), M2 protein (pH activated ion channels), and neuraminidase (NA). Therefore, one or more small molecules or polymers that have an affinity for the hemagglutinin (HA), M2 protein, or neuraminidase (NA) can be appropriate for influenza virus isolation so long as these molecules or their derivatives can be coupled to a solid support without impairing their binding capacity. These molecules are immobilized onto a solid support, which are then placed in contact with clinical samples for a period of time.

The natural receptor for HA is sialic acid (N-acetyl-neuraminic acid). Therefore, sialic acid or its derivatives can be appropriate for capturing the viruses so long as they can be coupled to a solid support. Preferably, but not necessarily, these chemicals are modified such that they will not be cleaved by neuraminidase, which is also located on the viral membrane. If these HA binding chemicals can be cleaved by neuraminidase, another chemical or condition (e.g. assay at low temperature (e.g. at 4 degree)) that inhibits neuraminidase but do not interfere with HA binding to viruses can be present in the solution. Since neuraminidase inhibitors are expected to bind to the enzyme, these inhibitors or other neuraminidase binding chemicals can also be immobilized onto a solid support and used for capturing influenza viruses. Examples for this class of chemical include Neu5AC2en, oseltamivir and zanamivir and their derivatives (e.g. the hydrolysis product of oseltamivir, which results in the conversion of a CO₂C₂H₅ to a COOH group). Preferably, Neu5AC2en and zanamivir or their analogues are immobilized on solid phase support via their 7-position of the sialic acid type structure.

A third possible target for influenza virus capture is the M2 protein, which is a pH activated ion channel. There are small chemicals that bind to this protein, e.g., amantadine and rimantadine. If these chemicals can be modified and immobilized onto a solid support without interfering with their binding activity, they can also be used for isolation and concentration of influenza viruses.

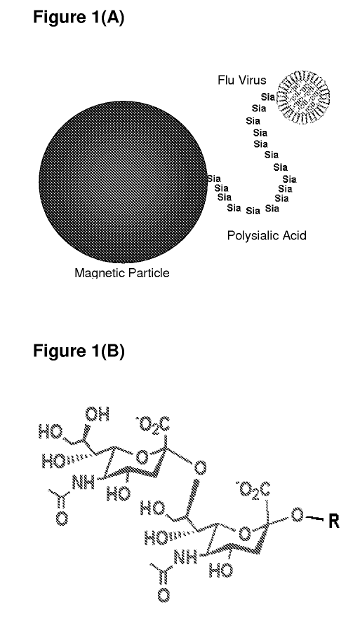

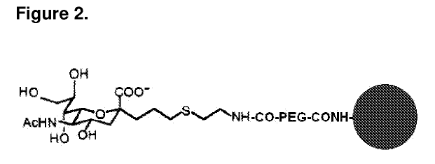

For the purpose of giving an example, a method for coupling polysialic acids onto magnetic particles is described below. Various applications using this reagent are described in subsequent sections. The reagent used in this example is best understood by referring to FIGS. 1A and 1B. FIG. 1A illustrates a magnetic particle coupled with polysialic acid that binds to an influenza virus whereas FIG. 1B shows the chemical structure of the poly(2, 8)sialic acid, a polymer composed of multiple units of sialic acid linked together via the 2, 8 linkage. FIG. 2 shows an example of C-glycoside of NA (N-acetylneuraminic acid) coated magnetic particle as virus capture reagent, which can be coated with O-glycoside or S-glycoside of N-acetylneuraminic acid as well. C-glycoside is preferred because it is neuraminidase-resistant. In FIG. 2 C-glycoside of N-acetylneuraminic acid is coated the magnetic particle via a flexible PEG (polyethylene glycol) linker. Examples of suitable glycosides can be found in J Med. Chem. 1993 Mar. 19; 36(6):778-83. J Med. Chem. 1995 Oct. 13; 38(21):4179-90. J Med. Chem. 1994 Sep. 30; 37(20): 3419-33. Another examples of NA derivatives include 4-alkyl or 7-alkyl or 4,7alkyl N-acetylneuraminic acids (e.g. those described in U.S. Pat. No. 6,303,764 and U.S. Pat. No. 6,420,552). The neuraminidase inhibitors described in U.S. Pat. No. 6,242,582 or 6,680,054 are also good candidates to be coated on the magnetic particles. Preferably they are coupled to the particle via their 7-position of the sialic acid structure.

### Preparation of an Influenza Virus Isolation Reagent

This reagent is a magnetic particle coated with polysialic acid. It is designed to bind to influenza viruses. Polysialic acid coupling can be performed as follows: 20 mg of amine coated magnetic particles (1.9 μm in diameter, Spherotech Inc. Libertyville, Ill.) are washed three times with 0.1 M MES, pH 5.0 and again three times with deionized water. The particle wet cake is suspended in 0.5 mL of polysialic acid (Sigma-Aldrich) at 20 mg/mL in deionized water, followed by an addition of 0.5 mL of 20 mg/mL carbodiimide [1-Ethyl-3-(3-dimethyl-aminopropyl)-carbodiimide hydrochloride, EDC] in deionized water, which is prepared immediately before use. The pH is then adjusted to 7.5 with 0.1 M NaHCO₃ solution. The particles were rotated at room temperature for 2 hours. Another 10 mg of EDC and 10 mg of NHS(N-hydroxysuccinimide) is added to the mix, followed by an overnight rotation at room temperature. The particles are washed 3 times with 10 mM HEPES buffer, pH 7.5, 5 times with deionized water and then suspended in 1.0 mL of deionized water. The reagent is now ready for use to capture the influenza viruses as described in the following applications.

### Detection of Influenza Viruses by Detecting Viral Nucleic Acids

Influenza viruses in a sample are isolated using the reagent. Viral RNA can be extracted from the viruses bound to the reagent and detected using an appropriate means such as PCR. Protocol examples for virus isolation, nucleic acid extraction and PCR detection are described below. It is understood that protocols specific for a sample may need to be optimized to achieve optimal results.

### Influenza Virus Isolation and Concentration

In this example, approximately 0.5 mg of the reagent is added to 1.0 mL PBS solution containing Influenza A viruses (A/Denver/57 from ATCC) with amounts ranging from 0.001 to 10 TCID₅₀ units. After 5 minutes incubation at room temperature, the reagent is attracted to the tube wall using a magnet and washed three times with PBS buffer. The influenza viruses in the samples are now captured and enriched on to the reagent.

### Influenza RNA Extraction and Detection

Viral RNA can be extracted directly from the bound viruses by suspending the reagent with viruses in a lysis buffer (e.g., one that is provided in the Qiagen Viral RNA Isolation Kit). The viral RNA is then isolated using the Qiagen kit according to the vendor's instructions. It is understood that other RNA extraction methods may also be appropriate for extracting influenza virus RNA from the bound reagent. For example, viruses can be lysed using a lysis buffer containing proteinase K and/or nonionic detergent such as Triton X-100. The viral lysate can be directly used for RT-PCR detection or used for detection after heat-inactivation (e.g., 94° C. for 10 minutes) if proteinase K is present in the lysis buffer.

The eluted RNA can used for RT-PCR detection using a primer set for the neuraminidase gene (forward primer: TCTGACCCAAGGCGCTCTGTTAAA (SEQ ID NO: 1); reverse primer: TCGGGCCATCGGTCATTATGGTAA (SEQ ID NO: 2)), followed by another round of nested PCR (forward primer: AATGAGCTGTCCTATCGGTGAAGC (SEQ ID NO: 3); reverse primer: ATACAGCCACTGCTCCATCATCTG (SEQ ID NO: 4)). The PCR products are resolved by agarose gel electrophoresis and visualized using ethidium bromide staining. The photograph of the gel in FIG. 2 shows that as little 0.001 TCID₅₀ units can be detected, indicating that the reagent is highly efficient in isolating and concentrating the influenza viruses.

2. Detection of Influenza Viruses by Detecting Viral Proteins (Antigens)

Influenza viral proteins can also be extracted from the isolated viruses and used for immunoassays. Methods for immunoassays are available in numerous published articles, books or internet information sites. In general, influenza viral proteins (antigens) can be captured on to a solid support that is coated with an antigen specific antibody. The captured antigen can be detected using another antibody that is conjugated with a detectable marker, e.g., horseradish peroxidase.

3. Detection of Influenza Viruses by Detecting Viral Neuraminidase Activity

Influenza viruses contain neuraminidase on the membrane, which can be detected using an appropriate substrate, which releases detectable signal (e.g., light) upon cleavage of the substrate by neuraminidase, such as that described by Achyuthan et. al. (Luminescence 18: 131-9; 2003).

For this application, the captured viruses should be dissolved with a solution that does not inactivate the enzyme. Appropriate solutions include, but are not limited to, PBS buffer with nonionic detergent (e.g., 1% Tween 20) and protease inhibitors. Alternatively, intact viruses can be released, as described below, and used in neuraminidase-based assays.

4. Detection of Influenza Viruses by Virus Culturing and Subsequent Detection

In certain applications, influenza virus culture is preferred because of the necessity to recover virus isolate for further analysis and/or for vaccine production. If the bound influenza viruses can be released under mild condition, e.g., in the presence of free binding chemical such as free polysialic acid, the released viruses may be cultured in eggs or culture cells.

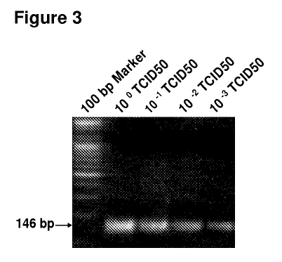

Our experiment showed that the bound viruses could be readily released in the presence of free polysialic acid. In this experiment, influenza viruses (100 TCID₅₀ units) in 1.0 mL PBS were incubated with 0.5 mg capture reagent for 5 minutes. The reagent with bound viruses was washed three times with PBS and then incubated with 50 μL of polysialic acid (PSA) at 100 or 1000 ng/mL for 10 minutes. The reagent was then removed using a magnet. The supernatant was subjected to RNA extraction using the Qiagen RNA isolation kit. 1/10 of the isolated RNA was used for one round of RT-PCR detection. The results shown in FIG. 3 indicate that the bound viruses could be efficiently released from the magnetic particles when 1000 ng/mL PSA (polysialic acid) was used as the releasing agent; 100 ng/mL PSA was less effective for releasing the bound viruses.

The fact that the viruses could be released with a hemagglutinin-binding competitor suggests that these viruses were still intact. This also suggests that the substrate bound to the viruses could be readily exchanged with other substrate molecules, indicating that the released viruses, which should be bound with the free polysialic acid, can be cultured because polysialic acid bound to the viruses could be exchanged with the sialic acids on cell surface, permitting productive infection.

Thus, influenza isolation reagents prepared according to the present invention can result in influenza viruses that could be cultured, which allows the establishment of a virus isolate. Once cultured, the viruses can be subjected to a range of testing such as HA neutralization assay and NA neutralization assay etc. to establish the subtype identity of the isolated viruses.

5. Direct Detection of Influenza Viruses Captured on Magnetic Particles

The bound influenza viruses can be directly detected without being eluted using a number of methods. For example, magnetism itself can be used as a signal for detection using a magnetism detection system such as that used in the Bead Array Counter (BARC) system [J C Rife et. al., Sensors Actuators A 107, 209-218 (2003)]. For this type of assay, the influenza isolation reagent is incubated with a sample containing influenza viruses under appropriate conditions. The reagent, now bound with influenza viruses if the viruses are present in the sample, is washed with an appropriate solution such as PBS.

The reagent-influenza virus complex is run through a solid support coated with an antibody specific for influenza surface antigen such as the HA and/or NA antigen. Magnetic particles with bound influenza viruses are captured while the free particles are washed away. The solid support can be microwells and microfluidic cartridge or other types. The retained magnetic particles can be detected with a magnetism detector such as giant magnetoresistive (GMR) sensor, superconducting quantum interference devices (SQUIDs), anisotropic magnetioresistive (AMR) rings, and miniature Hall crosses.

6. Use of the Influenza Virus Isolation Reagent for Influenza Virus Surveillance

It is well established now that water fowls, including ducks, are the natural reservoir of influenza viruses. The currently on-going H5N1 avian flu virus is believed to be residing in certain water fowls. Surveillance of this and other viruses in animals is important for monitoring and containing this virus and other avian flu viruses.

The common used surveillance methods involve taking samples from birds, including collecting tracheal swabs, cloacal swabs, or feces. Generally these methods result in very low (˜1%) “isolation rate”, i.e., the percentage of samples that result in recovery of virus isolates in an infected bird population. A number of factors may contribute to the low isolation rate. One of these factors may be that the samples are heavily contaminated with other materials (e.g., those in the feces) that may adversely affect the survivability of the viruses during shipping and culture efficiency.

The influenza isolation reagents disclosed in the present invention can be used to isolate and elute intact viruses, which can then be stored in appropriate medium for shipping and for culture. The isolated viruses can improve the isolation rate.

A protocol example is as follows: the samples collected from animals (e.g., tracheal swabs, cloacal swabs, or feces) are suspended in approximately 2.5 mL Hanks balanced salt solution, which is available from various vendors and the composition of which is available from published literature. It may be necessary to filter or briefly centrifuge to remove large debris, particularly those in the feces samples. Influenza virus isolation reagent is then added to the solution, mixed and incubated for appropriate amounts of time, e.g., 10 minutes. The reagent, now bound with the viruses, is washed several times with the same solution. The bound viruses can be eluted using a small amount (e.g., 100 μL) of Hanks balanced salt containing 1 μg/mL polysialic acids (or other appropriate competitor chemicals). The reagent is removed using a magnet. The eluted viruses are mixed with equal volumes of 2× transport medium (e.g., 2× Hanks Balanced Salt Solution supplemented with 20% glycerol, 400 Units/mL penicillin, 0.4 mg/mL streptomycin, 200 units/mL polymyxin B sulfate, and 0.5 mg/mL gentamycin). The recovered viruses can be used to inoculate embryonic eggs or culture cells.

7. Influenza Virus Isolation from Water as a Surveillance Method

Current animal surveillance methods use samples collected from individual animals. Because of the low isolation rate of these methods, they are highly inefficient and insensitive in detecting infected populations of birds, particularly in places like China, where there are billions of domestic ducks and geese in any given time. Therefore, there is a need for more efficient and sensitive surveillance method.

The present invention discloses a surveillance method that detects circulating influenza viruses at the population level, i.e., by collecting and detecting one sample from each natural population of ducks or geese or migratory birds. The sampling method involves collecting a large volume (e.g., 100 mL) of water from multiple spots of the water system on which the birds reside. The influenza viruses, if present in the water sample, is isolated and enriched using an influenza virus isolation reagent such as those described above. Influenza viral RNA can be extracted from the isolated viruses and used for RT-PCT detection. The water system that has detectable influenza viruses by RT-PCR method can be subjected to larger scale virus isolation using the same reagent to recover viruses that can be confirmed by culture methods. In addition, the birds that reside on the water system with detectable influenza viruses can be more intensively sampled for virus isolation and detection by culture methods.

There are several reasons that the water samples, if properly collected from the water systems where large flocks of ducks or geese reside, are a better source of influenza viruses for surveillance purposes. First, influenza viruses could be readily isolated from even unconcentrated water, as disclosed in a number of publications (e.g., Stallknecht et al. Avian Dis. 1990; 34: 406-411), because ducks/geese spend considerable amounts of time on the water and shed large amounts of viruses into the water. Second, influenza viruses can survive for a long period of time in water—up to 207 days at 17° C. and even longer at 4° C. (Stallknecht et al. Avian Dis. 1990; 34: 412-418)—which means that influenza viruses can accumulate over time in water systems. Third, ducks and geese often congregate as flocks, which can concentrate the viruses in certain spots in a water system. Fourth, a single sample can be collected as a mixture of water from multiple spots in a water system. Finally, water samples are far easier to process and should have fewer amounts of contaminants that inhibit PCR.

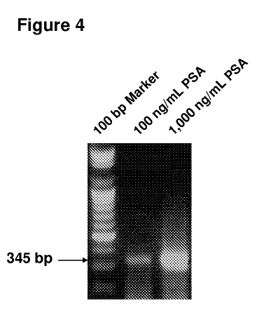

A protocol example is as follows: 100 mL of water is collected from each of 5 different spots of a water system on which a bird population (e.g., a flock of domestic ducks) resides. The water samples are pooled together to result in 500 mL of water sample. 100 to 200 mg of virus isolation reagent is mixed with the water sample for an appropriate time, e.g., 20 minutes. It is preferred that continuous mixing is carried out during the incubation using a device similar to that depicted in FIG. 4A. After incubation, the water sample is drained through a drainage tube that is enclosed on the outside with a magnet, or using a magnet clamp similar to that depicted in FIG. 4B. The retained reagent is washed into a small container such as a 50-mL conical tube. The reagent is washed a few more times using a magnet and Hanks balanced salt solution. The bound viruses can be eluted by incubating with a small volume (e.g., 200 μL) of Hanks balanced salt solution supplemented with 1 μg/mL of polysialic acid. The eluted viruses can be used for nucleic acid extraction and detection or for culture as described above. If transport is necessary, the eluted viruses are diluted with an equal volume of transport medium described above. Alternatively, nucleic acids can be extracted from the bound viruses and used for RT-PCR based detection.

### Example 2

### HIV Virus Isolation and Detection

Each year in the United States, approximately $10 billion was spent on acquired immunodeficiency syndrome (AIDS) caused by human immunodeficiency viruses (HIV) (source: Committee on Prevention and Control of Sexually Transmitted Diseases). Studies indicate that AIDS and other sexually transmitted diseases have reached an epidemic proportion. In Africa, HIV/AIDS infection has become a life and death issue not only for individuals, but also for entire nations. Battling HIV/AIDS has become a global priority.

The availability of cocktail chemotherapy for HIV infected patients has greatly improved the prospect of these patients. Administering of this chemotherapy requires close monitoring of HIV concentration in blood—commonly known as HIV viral load—as a tool to measure the effectiveness of the therapy and to reduce the chance of emergence of drug resistant viruses. During chemotherapy, many patients achieve very low HIV viral load, e.g., at hundreds RNA copies per mL serum or plasma samples. To detect very low viral load, it is critical to completely capture and concentrate the virus from biological samples, e.g. blood, without enriching contaminants that would inhibit downstream applications such as PCR detection. This is problematic because blood samples contain large amounts of PCR inhibitors such as heparins and heme-containing components (e.g., hemoglobin). Current method involves tedious and time-consuming ultracentrifugation using expensive instruments. It is therefore highly desirable to have a fast, inexpensive, easy-to-use method for isolating and concentrating HIV virus from biological samples.

The present invention involves the use of magnetic particles coated with HIV capture ligand for isolating and concentrating HIV virus from varieties of sample sources. The preferred HIV capture ligands are poly anions that can bind to HIV virus, possibly via its gp120 protein. The poly anions include, but are not limited to, a copolymer of maleic acid and styrenesulfonic acid, a polymer of polyvinyl phthalate sulfate, sulfated polysaccharides (e.g. curdlan sulfate, dextrin sulfate, fucoidan, and pentosan polysulfate, dextran sulfate, heparin, heparin sulfate, carrageenan), polyvinylsulfate (PVS), and polyanethole sulfonate, their copolymers with acrylic acids and salts thereof. Most preferably these polymers have high density of sulfonic or sulfate functional groups. One example of polymers encompasses copolymers of maleic acid and styrenesulfonic acid. Another example of polymers encompasses polymers of polyvinyl phthalate sulfate, which can be mixed esters comprising phthalate and sulfate functional groups on a polyvinyl backbone, and which can be produced as an esterification product of polyvinyl alcohol by phthalic anhydride and sulfuric chloride. Each of these classes of compounds has a high density of acid functional groups. Besides the ligand described above, there are also small molecule or polymer based HIV binding ligand from varieties of publications.

For example: cyclodextrin hexadecasulfate, PAVAS, PVAS, Suramin, SUC-HAS, ACO-HAS, ATA, JM3100 (listed in Journal of acquired immune deficiency syndromes and human retrovirology, 1996, 11, 211-221); T20, C34, ADS-J1, ADS-J2 (listed in Chem Bio Drug Des 2006; 67:13-26); Cyanovirin-N, mannose-specific plant lectins from Galanthus nivalis (GNA) and Hippeastrum hybrid (HHA), chicoric acid, vancomycin, teicoplanin, eremomycin, teicoplanin aglycon, BMS-378806, BMS-488043, AMD3100, AMD3465 (listed in Journal of medicinal chemistry, 2005, 48, 1297-1313). Other HIV capture ligand (e.g. some gp120 binding peptides) can also be used as long as it has affinity to HIV virus and can be immobilized on a solid phase support.

For copolymers of maleic acid and styrenesulfonic acid that are useful in the present invention, the molecular weight ratio of the maleic acid to the styrenesulfonic acid can be varied freely in almost any amount (e.g., molecular weight ratios are effective at from 9:1 to 1:9; 7:3 to 3:7; and at about 1:1). In one example, the molecular weight ratio of maleic acid to styrenesulfonic acid is about 1:3. The copolymers of maleic acid and styrenesulfonic acid (PSMA) can be made by well-known methods employing copolymerization of maleic acid with sulfonated styrene (e.g., Kobashi et al. U.S. Pat. No. 4,009,138), or by hydrolysis of a copolymer of maleic anhydrate and styrenesulfonic acid. The synthesis of copolymers of maleic anhydrate and styrenesulfonic acid is described by Bauman et al. (U.S. Pat. No. 2,835,655). They are also commercially available from Sigma-Aldrich, Inc.

These HIV capture ligand are immobilized on solid support to capture the virus. Preferably, the solid support is a magnetic particle. The advantages of using magnetic particles are discussed above.

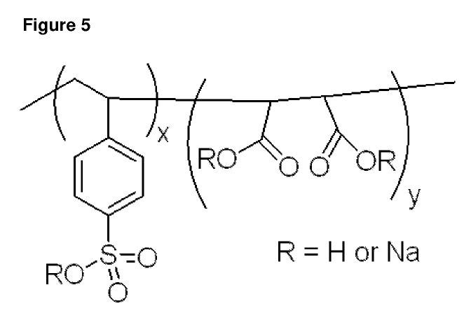

In one example, coupling of PSMA (FIG. 5) to the magnetic particle can be performed as follows: 20 mg of amine coated magnetic particles (1.9 μm in diameter, Spherotech Inc. Libertyville, Ill.) are washed three times with 0.1 M MES, pH 5.0 and again three times with deionized water. The particle wet cake is suspended in 0.5 mL of PSMA (Sigma-Aldrich) at 20 mg/mL in deionized water, followed by an addition of 0.5 mL of 20 mg/mL carbodiimide [1-Ethyl-3-(3-dimethyl-aminopropyl)-carbodiimide hydrochloride, EDC] in deionized water, which is prepared immediately before use. The pH is then adjusted to 7.5 with 0.1 M NaHCO₃ solution. The particles are rotated at room temperature for 2 hours. Another 10 mg of EDC and 10 mg of NHS(N-hydroxysuccinimide) are added to the mix, followed by an overnight rotation at room temperature. The particles are washed 3 times with 10 mM HEPES buffer, pH 7.5, 5 times with deionized water and then suspended in 1.0 mL of deionized water. The reagent is now ready for use to capture the HIV viruses. Similarly sulfated carbohydrate without carboxyl groups can be coated to the beads using suitable method accordingly (e.g. they can be coated to Dynabeads M-270 Epoxy, available from Dynal AS, Norway, using Dynal's coupling protocol).

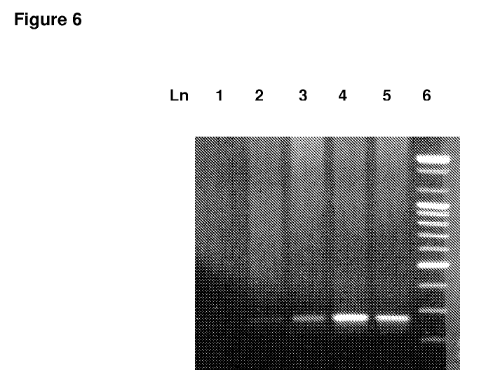

HIV-1 virus from the VQA HIV-1 standard (from NIH HIVAIDS Reagent Program) were spiked into approximately 1.0 mL human serum to a final concentration of 50, 100, or 500 viral RNA copies/mL. The samples were subjected to virus isolation using PSMA coated magnetic particles. 1/10 of the final viral lysate was used for RT-PCR followed by nested PCR. The amounts of viruses used for PCR were equivalent to 5, 10 and 50 viral RNA copies for the three samples, respectively. The PCR amplicons were resolved on an agarose gel and then visualized using ethidium bromide staining. As few as 50 HIV-1 RNA copies/mL could be isolated and detected. The results are shown in FIG. 6.

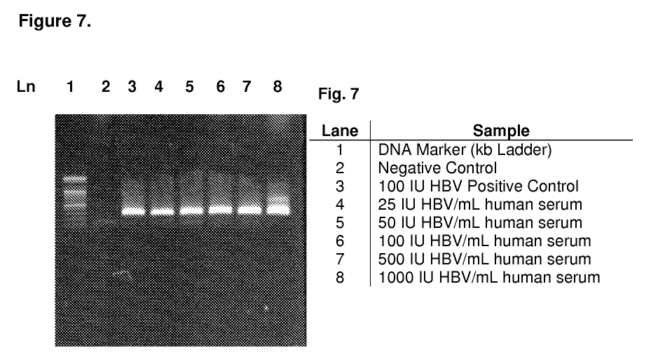

In FIGS. 7, 25 to 1000 IU HBV WHO standard were spiked to approximately 1 mL human serum and subjected to virus isolation using PSMA coated particles, using the same procedure as for HIV-1 isolation. The bound viruses were washed three times with 1 mL PBS and then lysed in a lysis buffer at 80-94° C. The viral lysate was directly used for nested PCR.

HIV RNA can be extracted from the captured HIV virus and detected with a RT-PCR method using protocols similar to those described for influenza viruses. It can also be used for PCR detection directly without nucleic extraction step. In one example, detection of HIV RNA involves the following steps: 1.0 mL of serum or plasma samples containing HIV viruses is mixed with 50 μl of 5 N NaCl, 50 μl of 1 M Tris-HCL (pH 8.0), and 1 mg of virus capture reagent (magnetic particles coupled with an appropriate polymer). After rotating at room temperature for 20 minutes, the particles (now bound with HIV viruses) are washed 3 times with 0.1×PBS using a magnet. The magnetic particles are suspended in 30 μl of elution buffer (20 mM Tris-HCl, pH 8.0, 1% Triton X-100 with or without Proteinase K at 1 mg/mL). If proteinase K is used in the lysis buffer, the particles mix is incubated in 50° C. water both for 1 hour, followed by heat inactivation (e.g., 95° C. for 10 minutes). If proteinase K is not used in lysis buffer, the reaction can proceed to heat inactivation without incubation at 50° C. An appropriate amount (e.g., 10 μl) can be directly used for RT-PCR detection.

### Example 3

### HCV Virus Isolation and Detection

Studies have shown that cellular binding of hepatitis C virus (HCV) envelope glycoprotein E2 requires cell surface heparin sulfate, a poly anionic chemical (Barth et al., J Biol Chem 278: 41003-41012; 2003). Therefore, HCV can be captured with heparin-coated particles. Coupling of heparin to magnetic particles can be accomplished using a method similar to coupling of PSMA to magnetic particles as described above.

HCV virus capturing and detection follow protocols that are similar to those for HIV or influenza viruses. The captured viruses can be directly used for RT-PCR as it is for HIV or influenza viruses.

### Example 4

### HAV and HBV Isolation and Detection

A reagent similar or identical to that used for HIV or HCV isolation and concentration can also be used for hepatitis A (HAV) or hepatitis B virus (HBV) isolation and concentration. HAV and HBV virus capturing and detection also follow protocols that are similar to those for HIV, HCV or influenza viruses.

It is understood that the methods disclosed in the present invention are appropriate for isolation and concentration of other viruses. For example, sialic acid is the native receptor for many viruses (e.g. SV40 virus, adenovirus, reovirus, paramyxoviruses); therefore these viruses can also be isolated with the flu capture reagent described above. Heparin has affinity for many viruses; these viruses include HCV, dengue virus, tick borne encephalitis virus, herpes simplex virus, papilloma virus and HIV. The PSMA coated particles can bind to HIV, HCV, and HBV and possibly other viruses that have not been tested.

### Example 5

### Culture of the Isolated Virus without Releasing the Virus from Capture Reagent

To culture the virus isolated from a sample with the capture reagent, the virus bound to the capture reagent can be first released from the capture reagent, followed by culturing with appropriate cells or tissues. However, the releasing step can be by-passed and, instead, the capture reagent with bound virus can be directly added to appropriate cells or tissue, resulting growth and recovery of the isolated virus.

For example, our experiment showed that the captured influenza virus could be cultured without being released from the capture reagent. The capture reagent bound with the virus could be added to cultured cells appropriate for influenza virus propagation. After incubation in appropriate conditions, influenza virus could be recovered from the cultured cell medium. Thus, it is not necessary to release influenza virus from the capture reagent for culture based detection or recovery of the virus. It is understood that this method of virus detection and recovery can be extended to other viruses as well.

### Example 6

### Use of Influenza Virus Capture Reagent in Vaccine Production

Virus binding reagents can be used as a virus purification tool during research, development and production of certain vaccines, therapeutics and diagnostic agents. For example, influenza virus binding reagent described in Example 1 can also be used for purifying influenza viruses during vaccine manufacturing. Purification of modified adenoviruses using virus binding reagent can be used in isolating adenoviruses containing gene therapy components. HIV viruses purified using the reagent described in Example 2 can be used to purify HIV viral antigens for diagnostic purpose such as those used in ELISA test kits. This example describes a method of using the influenza virus capture reagent for purifying flu viruses during flu vaccine production.

Crude influenza viruses produced in embryonated eggs or cultured cells are adjusted with NaCl and Tris buffer so that the solution contains 0.1 M NaCl and 50 mM Tris-HCl (pH 8.0), and then mixed with appropriate amounts of flu virus binding reagents. After mixing for 30 minutes, the particles are collected by filtering through an appropriate filtration system. After washing extensively to remove undesired substances, the bound viruses are eluted with an elution buffer with low pH (e.g., 50 mM glycine, pH 3.0).

Alternatively, the flu binding reagent is packaged into a column to make an affinity column for flu virus purification. Solutions containing influenza viruses are then passed through the column, which is then washed extensively to remove unwanted materials. The viruses bound to the column can be eluted with 50 mM glycine, pH 3.0. Other elution methods may also be used. For example, a chemical that competes with the influenza virus binding chemical coated on the solid support for the same binding site on the virus may be used to elute the virus.

It is understood that the solid support for preparing the virus binding reagent used in manufacturing of vaccines or therapeutics may require unique characteristics, i.e., the solid support is of pharmaceutical grade. For example, the solid support should have no or minimal amounts of leachable substances. The reagent should provide a large surface binding area to provide maximal binding capacity while providing easy passage wash solution for efficient removal of unbound contaminants. In addition, the column should permit easy passage of eluted virus with no or minimal physical hindrance. A variety of columns have been used in producing biopharmaceuticals. Some of the solid supports in these columns may be appropriate for applications described above.

### Example 7

### Use of Virus Binding Reagents for Viral Clearance during Biopharmaceutical Manufacturing

Processes designed to remove endogenous and exogenous viruses, also known as viral clearance, are critical for biopharmaceutical manufacturing. These biopharmaceuticals include those derived from human plasma, which are known as plasma derivatives such as human immunoglobulin.

The virus binding reagents described in the present invention can be used for viral clearance during biopharmaceutical manufacturing. When used as a combination of several different virus binding reagents, a broad spectrum of viruses can be removed from a biopharmaceutical preparation. For example, the sialic acid coated particles can bind viruses using sialic acid as the receptor; these viruses include influenza viruses, SV40, adenovirus, reovirus and paramyxoviruses whereas the heparin coated particles can bind to HCV, dengue viruses, tick borne viruses, herpes simplex viruses, papilloma viruses and HIV viruses, which can be removed with heparin coated reagents. The PSMA coated particles can bind to HIV, HBV and, possibly other viruses that have not been tested. Therefore, the use of a combination of all three reagents will remove a broad spectrum of viruses.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions. All publications and patents cited above are incorporated herein by reference.

What is claimed is:

1. A viral-particle isolation reagent, comprising:
(a) virus-particle binding agent and (b) a solid support, wherein said binding agent is coupled to said solid support and is capable of specifically binding to a binding partner present in a virus particle.

2. A method for isolating a virus, comprising contacting virus particles with a viral-particle-isolation reagent under conditions effective for said reagent to specifically bind to said virus particles, wherein said reagent comprises a virus-particle-binding agent which is coupled to a solid support and which is capable of specifically binding to a binding partner present in said virus particles.

3. A method of claim 2, wherein said virus-particle-binding-agent comprises polysialic acid or a derivative thereof.

4. A method of claim 2, wherein said virus-particle-binding agent is capable of specifically binding to a virus particle neuraminidase protein.

5. A method of claim 2, wherein said virus particle is present in a body fluid sample.

6. A method of claim 2, wherein said virus particle is present in an environmental sample.

7. A method of claim 2, further comprising eluting said virus particles from said reagent, and detecting neuraminidase activity present in said virus particles.

8. A method of claim 2, further comprising contacting said reagent with a viral binding partner capable of specifically binding to said viral particle, wherein said binding partner is coupled to a solid substrate which is utilized to capture said reagent bound to said virus.

9. A method of claim 8, wherein the solid support is a magnetic particle, and the presence of viral particles is determined by detecting the presence of magnetic material specifically bound to said binding partner.

10. An HIV capture reagent, comprising (a) a polyanion and (b) a solid support, wherein said polyanion is coupled to said solid support and is capable of specifically binding to a surface binding partner on an HIV viral particle.

11. An HIV capture reagent of claim 10, wherein said polyanion is a copolymer of maleic acid and styrenesulfonic acid, a polymer of polyvinyl phthalate sulfate, a sulfated polysaccharide, polyvinylsulfate, and polyanethole sulfonate, their copolymers with acrylic acids and salts thereof.

12. An HIV capture reagent of claim 11, wherein said sulfated polysaccharide is curdlan sulfate, dextrin sulfate, fucoidan, and pentosan polysulfate, dextran sulfate, heparin, heparin sulfate, or carrageenan.

13. A Hepatitis virus capture reagent, comprising (a) a polyanion and (b) a solid support, wherein said polyanion is coupled to said solid support and is capable of specifically binding to a surface binding partner on a Hepatitis viral particle.

14. A Hepatitis virus capture reagent of claim 13, wherein said polyanion is heparin sulfate.

15. A Hepatitis virus capture reagent of claim 14, wherein said Hepatitis viral particle is Hepatitis C virus.

16. A Hepatitis virus capture reagent of claim 14, wherein said Hepatitis viral particle is Hepatitis B virus or Hepatitis A virus.

17. A Hepatitis virus capture reagent of claim 13, wherein said polyanion is curdlan sulfate, dextrin sulfate, fucoidan, and pentosan polysulfate, dextran sulfate, heparin, heparin sulfate, carrageenan, copolymer of maleic acid and styrenesulfonic acid, polymer of polyvinyl phthalate sulfate, sulfated polysaccharide, polyvinylsulfate, or polyanethole sulfonate, or a copolymer thereof with acrylic acids or a salt thereof.

18. A method of purifying a virus from a biological or an environmental solution comprising treating said solution with a reagent of claim 1.

19. A method of removing a virus from a biological or an environmental solution comprising treating said solution with a reagent of claim 1.

20. A column packed with a reagent of claim 1.

21. A method of purifying a virus from a biological or an environmental solution comprising (a) passing the virus-containing solution through a column of claim 20, (b) washing the column to remove unwanted materials, and (c) eluting the bound virus.

22. A method of detecting a virus from a biological or an environmental specimen comprising (a) treating said specimen with a reagent of claim 1 and (b) releasing the nucleic acid from the virus bound to the said reagent and (c) directly detecting the released nucleic acid using polymerase chain reaction technique without nucleic acid extraction step.

23. A method of detecting and recovering a virus from a biological or an environmental specimen comprising (a) treating said specimen with a reagent of claim 1, (b) directly contacting the reagent resulted from step (a) without releasing the virus with cultured cells or tissue suitable for propagation of the said virus, and (c) detecting and recovering the said virus from the culture.


SEQUENCE LISTING (4 sequences)

SEQ ID NO: 1 - DNA, 24 bp, Artificial Sequence
  Description of Artificial Sequence Synthetic primer
tctgacccaaggcgctctgttaaa

SEQ ID NO: 2 - DNA, 24 bp, Artificial Sequence
  Description of Artificial Sequence Synthetic primer
tcgggccatcggtcattatggtaa

SEQ ID NO: 3 - DNA, 24 bp, Artificial Sequence
  Description of Artificial Sequence Synthetic primer
aatgagctgtcctatcggtgaagc

SEQ ID NO: 4 - DNA, 24 bp, Artificial Sequence
  Description of Artificial Sequence Synthetic primer
atacagccactgctccatcatctg